SEQUENCE LISTING (2 sequences)

SEQ ID NO: 1 - protein, 30 aa, Artificial sequence
  GALA peptide
WEAALAEALAEALAEHLAEALAEALEALAA

SEQ ID NO: 2 - protein, 30 aa, Artificial sequence
  R-GALA peptide
AALAELAEALAEALHEALAEALAEALAAEW

(19) United States
(12) Patent Application Publication
(10) Pub. No.: US 2015/0140066 A1
(43) Pub. Date: May 21, 2015

(54) CARRIER FOR INTRACELLULAR DELIVERY OF FUNCTIONAL PROTEIN

(71) Applicant: NATIONAL UNIVERSITY CORP. HOKKAIDO UNIVERSITY, Hokkaido (JP)

(72) Inventors: Hideyoshi Harashima, Hokkaido (JP); Yuma Yamada, Hokkaido (JP); Sandra Milena Vergara Perez, Hokkaido (JP); Yukari Yasuzaki, Hokkaido (JP)

(21) Appl. No.: 14/385,634

(22) PCT Filed: Sep. 19, 2012
(86) PCT No.: PCT/JP2012/073938

§ 371 Date: Jan. 26, 2015

(30) Foreign Application Priority Data
Mar. 21, 2012 (JP) 2012-063815

(51) Int. Cl. A61K 9/127; C07K 16/00; A61K 47/42

(52) U.S. Cl. CPC A61K 9/1271; A61K 47/42; C07K 16/00; C07K 2317/77
USPC 424/450; 514/1.1; 424/130.1

(57) ABSTRACT

A liposome comprising a lipid covalently bonded with a polyarginine peptide consisting of 4 to 20 continuous arginine residues and a lipid covalently bonded with a GALA peptide consisting of the amino acid sequence of SEQ ID NO: 1 and/or an R-GALA peptide consisting of the amino acid sequence of SEQ ID NO: 2 as component lipids of lipid membrane, and having a lipid membrane in which a protein to be intracellularly delivered is non-covalently bound to outer surface of the membrane, which is for quickly and conveniently deriver a functional protein, especially such a protein having a high molecular weight, into a cell.

### TECHNICAL FIELD

The present invention relates to a liposome carrier for intracellular delivery of a functional protein.

### BACKGROUND ART

Techniques for intracellularly delivering a functional protein having a high molecular weight such as antibodies attract strong commercial and scientific interests. In particular, intracellular delivery of an antibody that acts on a protein exhibiting a physiological function in a cell to control that function can notably expand choices of target molecules of so-called antibody drugs. Further, intracellular delivery of a functional protein that interacts with an intracellular biological molecule as a target, which does not depend on genetic recombination, can give important findings in researches of the molecular biology.

Some methods are known for intracellularly delivering a functional protein such as antibodies. One of them is covalently bonding an antibody and a functional peptide such as protein transduction domains (PTDs) and cell penetrating peptides (CPPs) based on genetic recombination or chemical synthesis. For example, an antibody covalently bonded to the PTD peptide consisting of 17 amino acid residues originating in the signal sequence of Kaposi fibroblast growth factor is delivered into fibrocytes (Non-patent document 1). However, such methods require time for the preparation of materials to be used, and also require chemical modification accompanied by a risk of changing binding characteristics of the antibody.

A simpler method is use of a modified CPP having a function of interacting with a protein to form a non-covalent complex with maintaining cell permeability (Non-patent document 2). Some of such peptides are commercially available reagents for intracellularly delivering a protein. However, as several reports have been made on accumulation of a CPP-protein complex in endosome, insufficient escape of the protein from the endosome to the cytoplasm constitutes a problem of the use of CPPs. Further, if based on the general understanding that a peptide is easily decomposed by proteases widely existing in living bodies, it is difficult to expect the same delivery efficiency as that obtainable at the time of applying such a method as mentioned above to culture cells, for application of the same to a living body (animal).

The use of HVJ-E (Hemaglutinating virus of Japan envelope), which is a deactivated Sendai virus particle, is an approach different from the use of a peptide (Non-patent document 3). However, the use of the virus still makes application thereof to a medicament difficult, even if it is deactivated.

One of examples of delivery of a protein using a non-virus vector is use of a cationic lipid, which non-covalently bonds to an antibody to form a complex. Such a lipid is marketed as reagents for research with trade names of “Lipodin-Ab” and “Ab-DeliverIN.” However, many of cationic lipid carriers used therein may be toxic for cells or living bodies, and therefore it is difficult to apply the aforementioned cationic lipid carrier to treatment of a disease, which requires long term administration of a functional protein. Further, use of such lipids also suffers from the problem of insufficient escape of a protein from the endosome to the cytoplasm. Furthermore, the time required for the intracellular delivery of a protein, i.e., several hours, also obstructs use of cationic lipids.

Liposome is one form of use of lipids, and is a delivery carrier that can be administered to a living body for delivering a nucleic acid such as siRNA into a cell, of which development has been continued even so far today. Liposome is a spherical particle having an internal space closed by a lipid bilayer membrane, and in many cases, a substance to be intracellularly delivered is encapsulated in the internal space. This is because the substance to be delivered encapsulated by a lipid bilayer membrane can obviate attacks by various biological substances including nucleases. However, on the other hand, it is required that the encapsulated substance is released in the inside of a cell through a process that the liposome is intracellularly incorporated into endosome through endocytosis or the like, the liposome escapes from the endosome to the cytoplasm, and further the encapsulated substance is released in the inside of the cell due to destruction of the lipid bilayer membrane by a certain means. Therefore, intracellular delivery of a substance using a liposome carrier faces a problem that the efficiency of each step, i.e., the intracellular incorporation by endocytosis or the like, escape of the liposome from the endosome to the cytoplasm, and the release of the encapsulated substance into the inside of the cell, should be increased. As for the intracellular delivery of a functional protein using a liposome encapsulating a functional protein, this problem should also be solved.

There has been reported intracellular delivery of a functional protein in which the protein is electrostatically bonded to the surface of liposome instead of encapsulation in the internal space of liposome (Patent document 1). However, this delivery requires binding a linker such as polynucleotide to the functional protein, thus the operation is complicated, and there is also a problem that the functional protein to be delivered is no longer the naturally existing protein.

Some of the inventors of the present invention constructed a liposome using a lipid bound to a peptide called GALA (henceforth referred to as GALA peptide) as a membrane component, and demonstrated that this liposome had superior property for the escape from the endosome, and enabled release of encapsulated substance into the cytoplasm (Patent document 2). However, this liposome is designed for intracellular delivery of the encapsulated substance. Further, they also constructed a liposome using a lipid bound to the GALA peptide and a lipid bound to a polyarginine peptide as membrane components, and reported that this configuration imparts resistance to biological components to the liposome (Patent document 3). However, this liposome is also designed for delivery of the encapsulated substance into a cell.

### PRIOR ART REFERENCES

### Patent Documents

Patent document 1: Japanese Patent Unexamined Publication (Kohyo) No. 2003-531820
Patent document 2: Japanese Patent Unexamined Publication (Kokai) No. 2006-28030
Patent document 3: International Patent Publication WO2008/105178

### Non-Patent Documents

Non-patent document 1: Y. Zhao et al., J. Immunol. Methods, 2001, Vol. 254, pp. 137-145
Non-patent document 2: M. C. Morris et al., Nat. Biotech., 2001, Vol. 19, pp. 1173-1176
Non-patent document 3: Y. Kondo et al., J. Immunol. Methods, 2008, Vol. 332, pp. 10-17

### SUMMARY OF THE INVENTION

### Object to be Achieved by the Invention

An object of the present invention is to provide a liposome having superior efficiency for intracellular delivery of a functional protein, which is a type of liposome without encapsulation of a functional protein in an internal space.

### Means for Achieving the Object

The inventors of the present invention found that a liposome utilizing a lipid bound to the GALA peptide and a lipid bound to a polyarginine peptide as component lipids, in which a functional protein to be intracellularly delivered is non-covalently bound directly to the outer surface of the lipid membrane of the liposome, achieves highly efficient delivery of the functional protein into a cell, and as a result, accomplished the present invention described in (1) to (6) mentioned below.

(1) A liposome comprising a lipid covalently bonded with a polyarginine peptide consisting of 4 to 20 continuous arginine residues and a lipid covalently bonded with a GALA peptide consisting of the amino acid sequence of SEQ ID NO: 1 and/or an R-GALA peptide consisting of the amino acid sequence of SEQ ID NO: 2 as component lipids of lipid membrane, and having a lipid membrane in which a protein to be intracellularly delivered is non-covalently bound to outer surface of the membrane.
(2) The liposome according to (1) mentioned above, wherein the polyarginine peptide, the GALA peptide and/or the R-GALA peptide, and the protein to be intracellularly delivered are disposed on the outer surface of one lipid membrane.
(3) The liposome according to (1) or (2) mentioned above, wherein the protein to be intracellularly delivered is a specific antibody directed to an intracellular protein.
(4) The liposome according to any one of (1) to (3) mentioned above, which is a single layer liposome comprising one lipid membrane.
(5) The liposome according to any one of (1) to (3) mentioned above, which comprises two or three lipid membranes.
(6) The liposome according to any of (1) to (5) mentioned above, which is for delivering a protein having a molecular weight larger than 1 kDa into cytoplasm or an intracellular organelle.

### Effect of the Invention

Use of the liposome of the present invention enables highly efficient and quick delivery of a functional protein non-covalently bound to the outer surface of the lipid membrane into a cell. Therefore, the liposome can intracellularly deriver an antibody of which target is a biological molecule existing in a cell, or a functional protein that interacts with such a biological molecule, while the antibody or functional protein maintains the physiological function thereof. Further, the liposome of the present invention does not require the step of encapsulating a functional protein into an internal space of the liposome for the preparation thereof, and this characteristic feature not only simplify the preparation process, but also makes it possible to avoid inactivation of the functional protein, which frequently occurs in the encapsulating step. Further, the liposome of the present invention enables quick intracellular delivery of a functional protein, i.e., release of the functional protein in the inside of the cells in about several tens of minutes after administration.

### BRIEF DESCRIPTION OF THE DRAWINGS

FIG. 1 is a graph showing intracellular incorporation ability of the liposome of the present invention.

FIG. 2A is a confocal microscopic image of the HeLa cells into which DCGIgG, which is a control carrier not having the R8 peptide, was incorporated, and FIG. 2B is a confocal microscopic image of the HeLa cells into which DCG0RIgG, which is a control carrier not having the GALA peptide, was incorporated.

FIG. 3A is a confocal microscopic image of the HeLa cells into which DCG0RIgG not having the GALA peptide was incorporated, and FIG. 3B is a confocal microscopic image of the HeLa cells into which the liposomes DCG2RIgG of the present invention was incorporated.

FIG. 4A is a confocal microscopic image of the HeLa cells into which IgG^{Alexa488}-encapsulating type liposomes were incorporated, and FIG. 4B is a confocal microscopic image of the HeLa cells into which the liposomes DCG2RIgG of the present invention were incorporated.

FIG. 5 is a graph showing comparison of efficiencies for introducing a protein into cells of the liposome of the present invention, as well as Pro-Ject (registered trade name) and Chariot (registered trade name), which are commercial protein-introducing reagents.

FIG. 6 includes confocal microscopic images of the HeLa cells into which the liposome of the present invention, as well as Pro-Ject (registered trade name) and Chariot (registered trade name), which are commercial protein-inducing reagents, were incorporated.

FIG. 7 is a graph showing change of amount of antibodies introduced by the liposomes of the present invention into the HeLa cells with the passage of time.

FIG. 8 is a graph showing amount of incorporated antibodies and cell distribution of the same observed in flow cytometry analysis performed by incubating the HeLa cells introduced with the liposomes of the present invention for various times.

FIG. 9 depicts confocal microscopic images showing results of observation over time of intracellular delivery of antibodies attained with the liposomes of the present invention.

FIG. 10 depicts confocal microscopic images of the HeLa cells introduced with anti-NPC antibodies by using the liposomes of the present invention.

FIG. 11 depicts confocal microscope images of the HeLa cells introduced with anti-P-Akt antibodies by using the liposomes of the present invention. The upper left photograph shows a superimposed image, the upper right photograph shows nuclei (blue), and the lower left photograph shows IgG^{Alexa488} (green) bound to the anti-P-Akt antibodies.

FIG. 12 depicts confocal microscope images of the HeLa cells introduced with anti-STAT3 antibodies by using the liposomes of the present invention. The upper left photograph shows a superimposed image, the upper right photograph shows nuclei (blue), and the lower left photograph shows IgG^{Alexa488} (green) bound to the anti-STAT3 antibodies.

FIG. 13 depicts confocal microscope images of liver tissue of a mouse administered with DCG2ROIgG, which is the liposome of the present invention. The upper left photograph shows nuclei (blue), the upper right photograph shows IgG (green of Alexa488), the lower left photograph shows liver vessel endothelial cells (red of Alex 647), and the lower right photograph shows a superimposed image.

### MODES FOR CARRYING OUT THE INVENTION

The liposome of the present invention comprises a lipid covalently bonded with a polyarginine peptide consisting of 4 to 20 continuous arginine residues and a lipid covalently bonded with a peptide consisting of the amino acid sequence of SEQ ID NO: 1 and/or a peptide consisting of the amino acid sequence of SEQ ID NO: 2 as component lipids of lipid membrane, and has a lipid membrane in which a protein to be intracellularly delivered is non-covalently bound to outer surface of the membrane.

### (1) Polyarginine Peptide

The polyarginine peptide consisting of 4 to 20 continuous arginine residues used in the present invention (henceforth referred to as PAP) is one embodiment of the “peptide comprising a plurality of continuous arginine residues” described in Patent document 3 (International Patent Publication WO2005/032593). In the present invention, the number of the arginine residues is preferably 6 to 12, more preferably 7 to 10.

In the liposome of the present invention, PAP is covalently bonded at the N-terminus or C-terminus thereof to a lipid constituting the lipid membrane of the liposome, and when the lipid is inserted into the lipid membrane, PAP is disposed so that it is exposed on the outer surface of the lipid membrane. In the liposome of the present invention, so far that PAP is disposed so that it is exposed on the outer surface of the lipid membrane, PAP exposed on the internal surface of the lipid membrane may also exist.

The lipid, to which PAP binds at the N-terminus or C-terminus thereof, may be any lipid that can constitute the lipid membrane of the liposome, and examples include lipids having a saturated or unsaturated fatty acid group or cholesterol group having 10 to 20 carbon atoms, such as stearyl group, palmitoyl group, oleyl group, stearyl group, and arachidoyl group, phospholipids, glycolipids, long chain fatty alcohols such as sterol, phosphatidylethanolamine, and cholesterol, polyoxypropylene alkyl, glycerin fatty acid esters, and the like. Preferred lipids are stearic acid and cholesterol.

### (2) GALA Peptide

The GALA peptide used in the present invention is a functional peptide consisting of the amino acid sequence described in the non-patent document of T. Kakudo et al. (Biochemistry, 2004, Vol. 43, pp. 5618-5623). The GALA peptide has a function of promoting fusion of lipid membranes of liposomes having the GALA peptide on the surfaces of lipid membranes under acidic conditions. Further, the GALA peptide is also considered to have a function of making a liposome having the GALA peptide on the surface of lipid membrane release an encapsulating substance into a cytoplasmic fraction, after the liposome is incorporated into the endosome by endocytosis. In addition, the R-GALA peptide, which has an amino acid sequence corresponding to the amino acid sequence of the GALA peptide inversed from the C-terminus side to the N-terminus side, has the same function as that of the GALA peptide, and in the liposome of the present invention, the GALA peptide and the R-GALA peptide can be interchangeably or simultaneously used. Hereafter, the present invention will be explained by exemplifying the GALA peptide.

The number and position of amino acid residues to be deleted, substituted or added in the amino acid sequence of the GALA peptide is not particularly limited so long as the peptide (b) can fuse lipid membranes under acidic conditions, and the number of amino acid residues is one, or two or more, preferably one or several. Specifically, the number is usually in the range of 1 to 4, preferably 1 to 3, more preferably 1 or 2, for deletion, usually in the range 1 to 6, preferably 1 to 4, more preferably 1 or 2, for substitution, or usually in the range 1 to 12, preferably 1 to 6, more preferably 1 to 4, for addition.

In the liposome of the present invention, the GALA peptide is covalently bonded at the N-terminus or C-terminus thereof to a lipid constituting the lipid membrane of the liposome, and when the lipid is inserted into the lipid membrane, the GALA peptide is disposed so as to be exposed on the outer surface of the lipid membrane. In the liposome of the present invention, so long as the GALA peptide is disposed so as to be exposed on the outer surface of the lipid membrane, there may be the GALA peptide exposed on the internal surface of the lipid membrane.

The lipid, to which GALA peptide covalently bonds at the N-terminus or C-terminus thereof, may be any lipid that can constitute the lipid membrane of the liposome, and examples include lipids having a saturated or unsaturated fatty acid group or cholesterol group having 10 to 20 carbon atoms, such as stearyl group, palmitoyl group, oleyl group, stearyl group, and arachidoyl group, phospholipids, glycolipids, long chain fatty alcohols such as sterol, phosphatidylethanolamine, and cholesterol, polyoxypropylene alkyl, glycerin fatty acid esters, and the like. Preferred lipids are stearic acid and cholesterol. In addition, for both PAP and the GALA peptide, an amino acid residue such as cysteine residue, or an appropriate functional group may be added to the end thereof for covalently bonding it to a lipid or the like, and the peptide having such a group at the end thereof also falls within the scope of PAP or the GALA peptide.

### (3) Functional Protein

The functional protein non-covalently bound to the outer surface of the lipid membrane constituting the liposome of the present invention may be a protein intended to be delivered into the inside of a cell, and having a certain physiological activity. Examples include intracellular proteins that originally localize in cells to exhibit a certain function. If such an intracellular protein can be intracellularly delivered without relying on artificial genetic manipulation, it will become an effective research tool for molecular biological research of cells. In particular, it will be effective especially for cells for which establishment of host-vector system is insufficient.

Further, a protein expected to control a function of an intracellular protein through an interaction with the intracellular protein is also a preferred example of the protein intended to be intracellularly delivered by using the liposome of the present invention. Examples of such a protein include a specific protease that recognizes a specific amino acid sequence and hydrolyzes it, a nucleic acid binding enzyme that recognizes specific nucleotide sequences and binds them, and the like. A particularly preferred example is an antibody that specifically binds to an intracellular protein. Such an antibody is preferably a monoclonal antibody, most preferably a monoclonal antibody having the Fc region. Type of the antibody, such as IgG and IgM, and type of the intracellular protein as the object of the specific binding are not particularly limited. Further, the functional protein intended to be intracellularly delivered may be a protein having difficulty in spontaneous migration from the outside of a cell into the cytoplasm, and the molecular weight thereof is contemplated to be 1 kDa (kilo Dalton) or larger. In particular, a gigantic protein having a molecular weight of 100 kDa or larger, such as antibodies, and a protein complex formed from two or more molecules of protein are preferred examples of the functional protein to be intracellularly delivered referred to in the present invention.

### (4) Structure of Liposome

Number of the lipid membrane of the liposome of the present invention is not particularly limited so long as the liposome is a closed vesicle having a lipid membrane constituted by a lipid bilayer. It may be a multilamellar vesicle (MLU), or a single layer liposome, such as small unilamellar vesicle (SUV), large unilamellar vesicle (LUV), or giant unilamellar vesicle (GUV).

In the case of the single layer liposome, PAP, the GALA peptide, and the functional protein to be intracellularly delivered are disposed on the outer surface of the single membrane. Whilst, in the case of MLU, PAP, the GALA peptide, and the functional protein to be intracellularly delivered may be disposed on the outer surfaces of the respective lipid membranes, or may be selectively disposed on the outer surface of any of the lipid membranes. Further, PAP, the GALA peptide, and the functional protein to be intracellularly delivered may be placed on the outer surface of the same lipid membrane, or PAP and the GALA peptide, and the functional protein may be placed on the outer surfaces of different lipid membranes. In the case of MLV, PAP and the GALA peptide are preferably placed on the outer surface of the same lipid membrane. Further, in the case of MLV, the lipid membranes, especially the outermost lipid membrane, may be modified with another functional substance usable for liposome carrier, for example, a hydrophilic polymer such as polyalkylene glycol, a ligand specific to a target tissue or target cell, and others.

An example of MLV according to the present invention is a bilayer liposome consisting of an inner lipid membrane where PAP, the GALA peptide, and the functional protein to be intracellularly delivered are disposed on the outer surface thereof, and an outer lipid membrane surrounding the inner lipid membrane. Further, another example is a bilayer liposome consisting of an inner lipid membrane where the functional protein to be intracellularly delivered is disposed on the outer surface thereof, and an outer lipid membrane surrounding the inner lipid membrane, where PAP and the GALA peptide are disposed on the outer surface thereof. Furthermore, still another example of MLV according to the present invention is a trilayer liposome consisting of an innermost lipid membrane where the functional protein to be intracellularly delivered is disposed on the outer surface thereof, an intermediate lipid membrane surrounding the innermost lipid membrane, where the functional protein to be intracellularly delivered, which may be the same as or different from the functional protein of the innermost lipid membrane, is disposed on the outer surface thereof, and an outermost lipid membrane surrounding the intermediate lipid membrane, where PAP and the GALA peptide are disposed on the outer surface thereof. However, the liposome of the present invention is not limited to these examples.

In the internal space closed with the lipid membrane of the liposome of the present invention, a substance to be intracellularly delivered, which is different from the functional protein non-covalently bound to the surface of the lipid membrane, may be encapsulated. Type of the target substance is not particularly limited, and examples include, for example, medicaments, nucleic acids, peptides, proteins, saccharides, complexes of these, and the like. The target substance can be appropriately chosen depending on the purpose, such as diagnosis or treatment. The “nucleic acid” includes DNA and RNA, as well as analogues and derivatives thereof (for example, peptide nucleic acid (PNA), phosphorothioate DNA, and the like). The nucleic acid may be a single-stranded or double-stranded nucleic acid, and may be a linear or cyclic nucleic acid.

Although size of the liposome of the present invention is not particularly limited, it is preferably 50 to 800 nm in diameter, more preferably 100 to 200 nm in diameter.

### (5) Component of Lipid Membrane

Although type of the lipids constituting the lipid membrane of the liposome of the present invention is not particularly limited, phospholipids, glycolipids, sterols, long chain fatty alcohols, glycerin fatty acid esters, and the like can be used as the lipids including the lipids to which PAP or the GALA peptide covalently bonds.

Examples of the phospholipids include, for example, phosphatidylcholines (for example, dioleoylphosphatidylcholine, dilauroylphosphatidylcholine, dimyristoylphosphatidylcholine, dipalmitoylphosphatidylcholine, distearoylphosphatidylcholine, and the like), phosphatidylglycerols (for example, dioleoylphosphatidylglycerol, dilauroylphosphatidylglycerol, dimyristoylphosphatidylglycerol, dipalmitoylphosphatidylglycerol, distearoylphosphatidylglycerol, and the like), phosphatidylethanolamines (for example, dioleoylphosphatidylethanolamine, dilauroylphosphatidylethanolamine, dimyristoylphosphatidylethanolamine, dipalmitoylphosphatidylethanolamine, distearoylphosphatidylethanolamine (DSPE), dioleoylglycerophosphoethanolamine (DOPE), and the like), phosphatidylserine, phosphatidylinositol, phosphatidic acid, cardiolipin, hydrogenated compounds of these, natural lipids originating in yolk, soybean, other animals and plants (for example, yolk lecithin, soybean lecithin, and the like), and the like, and one or more kinds of these can be used. The aforementioned phospholipids are used as the main component of the lipid membrane. Amount thereof is preferably 10 to 100% (molar ratio), more preferably 50 to 80% (molar ratio), in terms of amount based on the total lipids of the lipid membrane structure, but the amount is not particularly limited by these values.

Examples of the glycolipids include sphingomyelins, glyceroglycolipids such as sulfoxyribosyl glyceride, diglycosyl diglyceride, digalactosyl diglyceride, galactosyl diglyceride, and glycosyl diglyceride, sphingoglycolipids such as galactosyl cerebroside, lactosyl cerebroside and ganglioside, and the like, and one or two or more kinds of these can be used.

Examples of the sterols include animal-derived sterols such as cholesterol, cholesterol succinate, lanosterol, dihydrolanosterol, desmosterol and dihydrocholesterol, plant-derived sterols (phytosterols) such as stigmasterol, sitosterol, campesterol and brassicasterol, microorganism-derived sterols such as thymosterol and ergosterol, and the like, and one or two or more kinds of these can be used. These sterols can be generally used for physically or chemically stabilizing the lipid bilayer or controlling fluidity of the membrane. Amount thereof is preferably 5 to 40% (molar ratio), more preferably 10 to 30% (molar ratio), in terms of amount based on the total lipids of the lipid membrane structure, but the amount is not particularly limited by these values.

As the long chain fatty acid or long chain fatty alcohol, a fatty acid or corresponding fatty alcohol having 10 to 20 carbon atoms can be used. Examples of such long chain fatty acid or long chain fatty alcohol include, for example, saturated fatty acids such as palmitic acid, stearic acid, lauric acid, myristic acid, pentadecyl acid, arachidic acid, margaric acid, and tuberculostearic acid, unsaturated fatty acids such as palmitoleic acid, oleic acid, arachidonic acid, vaccenic acid, linolic acid, linolenic acid, arachidonic acid, and eleostearic acid, oleyl alcohol, stearyl alcohol, lauryl alcohol, myristyl alcohol, cetyl alcohol, linolyl alcohol, and the like, and one or two or more kinds of these can be used. Amount thereof is preferably 5 to 40% (molar ratio), more preferably 10 to 30% (molar ratio), in terms of amount based on the total lipids of the lipid membrane structure, but the amount is not particularly limited by these values.

Examples of the glycerin fatty acid esters include monoacyl glycerides, diacyl glycerides, and triacyl glycerides, and one or two or more kinds of these can be used. Amount thereof is preferably 5 to 40% (molar ratio), more preferably 10 to 30% (molar ratio), in terms of amount based on the total lipids of the lipid membrane structure, but the amount is not particularly limited by these values.

Examples of the cationic lipids include, besides the lipids mentioned above, for example, dioctadecyldimethylammonium chloride (DODAC), N-(2,3-oleyloxyl)propyl-N,N,N-trimethylammonium (DOTMA), didodecylammonium bromide (DDAB), 1,2-dioleyloxy-3-trimethylammonium propane (DOTAP), 3β-N—(N′,N′-dimethylaminoethane)carbamol cholesterol (DC-Chol), 1,2-dimyristoyloxypropyl-3-dimethylhydroxyethylammonium (DMRIE), 2,3-dioleyloxy-N-[2-(sperminecarboxamido)ethyl]-N,N-dimethyl-1-propaneammonium trifluoroacetate (DOSPA), and the like, and one or two or more kinds of these can be used. Since cationic lipids have cytotoxicity, it is preferable to make the amount of the cationic lipid contained in the lipid bilayer as small as possible from the viewpoint of reducing the cytotoxicity of the liposome of the present invention. Ratio of the amount of the cationic lipid relative to the total lipids constituting the lipid bilayer is preferably 0 to 40% (molar ratio), more preferably 0 to 20% (molar ratio).

Examples of the neutral lipids include, besides the lipids mentioned above, for example, diacylphosphatidylcholines, diacylphosphatidylethanolamines, ceramides, and the like, and one or two or more kinds of these can be used. Examples of the anionic lipids include, besides the lipids mentioned above, for example, diacylphosphatidylserines, diacylphosphatidic acids, N-succinylphosphatidyl-ethanolamine (N-succinyl-PE), phosphatidylethylene glycol, and the like, and one or two or more kinds of these can be used.

Lipids are indispensable liposome membrane components, and amount of them is usually 50 to 100% (molar ratio), preferably 65 to 100% (molar ratio), more preferably 75 to 100% (molar ratio), of the total amount of the liposome membrane components.

In the aforementioned range, the ratio P of the lipids covalently bonded with PAP or the GALA peptide relative to the total amount of lipids constituting the lipid membrane, which is taken as 100(%), preferably satisfies the following condition: 5 mol %≦P≦25 mol %. Further, amounts of PAP and the GALA peptide existing on the surface of the liposome of the present invention relative to the total lipids constituting the lipid membrane of the liposome are usually 5 to 30 mol %, preferably 10 to 25 mol %, more preferably 15 to 20 mol %, for PAP, and usually 0.5 to 3 mol %, preferably 1.0 to 2.5 mol %, more preferably 1.5 to 2 mol %, for the GALA peptide.

In the liposome of the present invention, especially in the case of MLV, the surface of the outermost lipid membrane may be modified with a hydrophilic polymer. Type of the hydrophilic polymer is not particularly limited so long as it is a hydrophilic polymer that can improve the blood retention property of the liposome when the liposome is administered to a living body. Examples of such a hydrophilic polymer include polyalkylene glycols (for example, polyethylene glycol, polypropylene glycol, polytetramethylene glycol, polyhexamethylene glycol, and the like), dextran, pullulan, Ficoll, polyvinyl alcohol, styrene-maleic anhydride alternating copolymer, divinyl ether-maleic anhydride alternating copolymer, amylose, amylopectin, chitosan, mannan, cyclodextrin, pectin, carrageenan, and the like. Among them, polyalkylene glycols are preferred, and polyethylene glycol is more preferred. Preferred molecular weight of polyalkylene glycol is usually 300 to 10000, more preferably 500 to 10000, still more preferably 1000 to 5000.

Further, when the liposome of the present invention is MLV, a substance that can specifically binds to a tissue or cell to which the functional protein is to be delivered may be disposed on the surface of the outermost membrane. Although type of such a substance is not is particularly limited, examples include, for example, transferrin, insulin, folic acid, hyaluronic acid, an antibody directed to a biological molecule existing on a cell surface or a fragment thereof, sugar chain, growth factor, apolipoprotein, and the like.

In the present invention, the lipid membrane may contain, besides the lipids mentioned above, antioxidants such as tocopherol, propyl gallate, ascorbyl palmitate, and butylated hydroxytoluene, saturated or unsaturated fatty amines such as stearylamine and oleylamine, charged substances that impart positive charge, including saturated or unsaturated cationic synthetic lipids such as dioleoyltrimethylammonium propane, charged substances that impart negative charge such as dicetyl phosphate, cholesteryl hemisuccinate, phosphatidylserine, phosphatidylinositol, and phosphatidic acid, membrane stabilization agents, membrane proteins, and the like, and contents thereof can be appropriately adjusted.

The charged substances are optional liposome membrane components that can be added for imparting positive or negative charge to the liposome membrane, and amount thereof is usually 0 to 50% (molar ratio), preferably 0 to 30% (molar ratio), more preferably 0 to 20% (molar ratio), of the total amount of the liposome membrane components.

### (6) Method for Preparing Liposome

The liposome of the present invention can be prepared by preparing a carrier liposome having a lipid membrane containing a lipid covalently bonded with PAP and a lipid covalently bonded with the GALA peptide as component lipids, and mixing this carrier liposome and a functional protein to be intracellularly delivered in an appropriate buffer.

The carrier liposome can be prepared by using a known method such as the hydration method, ultrasonication method, ethanol injection method, ether injection method, reverse phase evaporation method, surfactant method, and freezing and thawing method. For example, in the case of the hydration method, a lipid bonded with PAP or the GALA peptide, other lipids, and optional components mentioned above to be contained in the lipid membrane are dissolved in an organic solvent, then the organic solvent is removed by evaporation to obtain a lipid membrane, and then the lipid membrane can be hydrated and subjected to stirring or ultrasonication to prepare a lipid membrane structure containing the lipid bonded with the peptide according to the present invention as a membrane component.

Further, the carrier liposome used in the present invention can also be prepared by dissolving the lipids mentioned above and other lipids in an organic solvent, then removing the organic solvent by evaporation to obtain a lipid membrane, hydrating the lipid membrane, subjecting the hydrated lipid membrane to stirring or ultrasonication to prepare a liposome, and then adding a lipid bonded with PAP or the GALA peptide to the liposome so that the peptide is introduced onto the outer surface of the liposome.

In addition, by passing the liposomes through a filter having a predetermined pore size, a lipid membrane structures having a certain particle size distribution can be obtained.

As the organic solvent, there can be used, for example, hydrocarbons such as pentane, hexane, heptane, and cyclohexane, halogenated hydrocarbons such as methylene chloride and chloroform, aromatic hydrocarbons such as benzene and toluene, lower alcohols such as methanol and ethanol, esters such as methyl acetate and ethyl acetate, ketones such as acetone, and the like, which can be used independently, or as a combination of two or more kinds of them.

When the substance to be enclosed in the internal space closed by the lipid membrane is a water-soluble substance, the substance can be enclosed in the internal space by adding the substance to the aqueous solvent used when the lipid membrane is hydrated in the preparation of the carrier liposome. Further, when the substance is a liposoluble substance, it can be enclosed in the lipid membrane of the liposome by adding the substance to the organic solvent used in the preparation of the carrier liposome.

By mixing the carrier liposome prepared as described above and a functional protein in an appropriate buffer, the liposome of the present invention is prepared. As the buffer used in this process, a buffer of a type suitable for maintaining activity of the functional protein can be selected depending on the type of the functional protein, and used.

When a functional protein is enclosed in an internal space of a liposome, the functional protein is inevitably subjected to ultrasonication at the time of the preparation of the carrier liposome by the hydration method, and such a process is accompanied by the risk of inactivation of the protein. According to the present invention, the functional protein need not be exposed to ultrasonication at the time of the preparation thereof, and this characteristic feature constitutes one of the advantages of the present invention.

Although the carrier liposome and the functional protein can be mixed at an arbitrary mixing ratio, the ratio of lipid:functional protein is preferably in the range of 1:0.1 to 5, more preferably 1:1. When a functional protein is encapsulated in the internal space of liposome, a lot of antibody solution is required at the time of the hydration. However, the present invention is more advantageous in terms of cost and simplicity of the process, for example, about 70% of the functional protein mixed with the carrier liposome binds with the lipid membrane surface.

The prepared liposome of the present invention can be stored and used in a state of a dispersion. As the dispersion solvent, for example, physiological saline, or a buffer such as phosphate buffer, citrate buffer, and acetate buffer can be used. For the dispersion, additives such as saccharides, polyhydric alcohols, water-soluble polymers, nonionic surfactants, anti-oxidants, pH modifiers, and hydration enhancers can be used by adding them to the dispersion. Further, the liposome of the present invention may be prepared immediately before use thereof by mixing the carrier liposome dried beforehand (for example, by lyophilization, spray drying, or the like) and a functional protein.

The biological species to which the liposome of the present invention can be administered is not particularly limited, and may be any of animal, plant, microorganism, and the like. However, it is preferably an animal, more preferably a mammal. Examples of the mammal include, for example, human, ape, bovine, ovine, goat, equine, swine, rabbit, canine, cat, rat, mouse, guinea pig, and the like.

The liposome of the present invention can be used in vivo (including administration to a living body), or in vitro. Examples of the route of administration of the liposome of the present invention to a living body include, for example, parenteral administration such as intravenous, intraperitoneal, subcutaneous, and nasal administrations, and dose and frequency of the administration can be appropriately adjusted according to the type, amount, purpose of the administration, and the like of the functional protein to be intracellularly delivered.

Hereafter, the present invention will be explained in a non-limitative manner with reference to examples and comparative examples.

### EXAMPLES

### Example 1

### Preparation of Liposome Bonded with IgG Antibody

### (1) Preparation of Carrier Liposome

According to the method described in Non-patent document 1 mentioned above, a C-terminus-amidated GALA peptide consisting of the amino acid sequence of SEQ ID NO: 1 was chemically synthesized by using a peptide synthesizer, purified, and then reacted with cholesterol to prepare cholesterylated GALA peptide (Chol-GALA). Similarly, according to the method described in Non-patent document 1 mentioned above, a C-terminus-amidated octarginine peptide (R8) consisting of eight arginine residues was chemically synthesized by using a peptide synthesizer, purified, and then reacted with stearic acid to prepare sterylated octarginine peptide (STR-R8).

DOPE/PA (molar ratio=9:2) modified with 0.55 mM rhodamine was put into four of glass test tubes, Chol-GALA was added to DOPE/PA so as to give a concentrations of 0 mol %, 1 mol %, 2 mol %, and 3 mol %, and a 1:1 mixture of ethanol and chloroform was further added to dissolve them (total volume=250 μL). Nitrogen gas was blown to the solution in each glass test tube to evaporate it to dryness to solidify the mixed lipids. A 10 mM HEPES buffer (pH 7.4, 250 μL) was added to the resultant to perform hydration for 10 minutes, and then the hydrated lipids were subjected to ultrasonication by using a water tank type sonicator to prepare multilamellar liposomes. The same operation was repeated with 0.55 mM DOPE/CHEMS (molar ratio=9:2).

To 0.55 mM multilamellar liposomes of each type, STR-R8 was added in an amount of 20 mol % based on the total lipids, and then the mixture was incubated at room temperature for 30 minutes to obtain carrier liposomes in which the R8 peptide and the GALA peptide were disposed on the outer surface of the outermost lipid membrane.

(2) Preparation of Liposomes Non-Covalently Bound with IgG Antibody

By using AlexaFluor (registered trade name) 488 Protein Labeling Kit (Invitrogen) according to the recommended protocol, goat IgG (Rockland) was labeled to obtain Alexa 488-labeled IgG antibody (henceforth referred to as IgG^{Alexa488}). Each type of the carrier liposomes (lipids, 0.55 mM) obtained in (1), and 0.125 mg/mL of IgG^{Alexa488} dissolved in the 10 mM HEPES buffer were mixed at a volume ratio of 1:1, and the mixture was incubated at room temperature for 15 minutes to obtain liposomes in which IgG^{Alexa488} non-covalently bound to the outer surface of the outermost lipid membrane. Sizes, PDIs, and ζ (zeta) potentials of the liposomes were measured by using Zetasizer Nano ZS ZEN3600 (MALVERN Instruments), and the results are shown in Table 1.

TABLE 1

| | GALA | Size | | | |
|---|---|---|---|---|---|
| | (mol %) | (nm) | PDI | ζ Potential | Abbreviation |
| DOPE/ | 0 | 132.1 | 0.242 | 41.2 | DPG0RIgG |
| PA | 1 | 146.5 | 0.196 | 31.6 | DPG1RIgG |
| | 2 | 146.2 | 0.155 | 27.0 | DPG2RIgG |
| | 3 | 151.1 | 0.145 | 23.6 | DPG3RIgG |
| DOPE/ | 0 | 184.5 | 0.401 | 52.9 | DCG0RIgG |
| CHEMS | 1 | 133.1 | 0.276 | 51.9 | DCG1RIgG |
| | 2 | 124.1 | 0.268 | 47.5 | DCG2RIgG |
| | 3 | 136.1 | 0.237 | 44.6 | DCG3RIgG |

The configurations of the liposomes can be described as follows.

### DPG0RIgG

Multilamellar liposome having a lipid composition of DOPE:PA:Chol-GALA:STR-R8=9:2:0:2.2 (molar ratio), and having R8 as PAP and IgG^{Alexa488} on the outer surface of the outermost lipid membrane

### DPG1RIgG

Multilamellar liposome having a lipid composition of DOPE:PA:Chol-GALA:STR-R8=9:2:0.11:2.2 (molar ratio), and having R8 as PAP, the GALA peptide, and IgG^{Alexa488} on the outer surface of the outermost lipid membrane

### DPG2RIgG

Multilamellar liposome having a lipid composition of DOPE:PA:Chol-GALA:STR-R8=9:2:0.22:2.2 (molar ratio), and having R8 as PAP, the GALA peptide, and IgG^{Alexa488} on the outer surface of the outermost lipid membrane

### DPG3RIgG

Multilamellar liposome having a lipid composition of DOPE:PA:Chol-GALA:STR-R8=9:2:0.33:2.2 (molar ratio), and having R8 as PAP, the GALA peptide, and IgG^{Alexa488} on the outer surface of the outermost lipid membrane

### DCG0RIgG

Multilamellar liposome having a lipid composition of DOPE:CHEMS:Chol-GALA:STR-R8=9:2:0:2.2 (molar ratio), and having R8 as PAP and IgG^{Alexa488} on the outer surface of the outermost lipid membrane

### DCG1RIgG

Multilamellar liposome having a lipid composition of DOPE:CHEMS:Chol-GALA:STR-R8=9:2:0.11:2.2 (molar ratio), and having R8 as PAP, the GALA peptide, and IgG^{Alexa488} on the outer surface of the outermost lipid membrane

### DCG2RIgG

Multilamellar liposome having a lipid composition of DOPE:CHEMS:Chol-GALA:STR-R8=9:2:0.22:2.2 (molar ratio), and having R8 as PAP, the GALA peptide, and IgG^{Alexa488} on the outer surface of the outermost lipid membrane

### DCG3RIgG

Multilamellar liposome having a lipid composition of DOPE:CHEMS:Chol-GALA:STR-R8=9:2:0.33:2.2 (molar ratio), and having R8 as PAP, the GALA peptide, and IgG^{Alexa488} on the outer surface of the outermost lipid membrane

### Example 2

### Confirmation of Incorporation of Liposomes into HeLa Cells and Intracellular Localization of Antibodies

### (1) Confirmation of Incorporation Efficiency

The liposomes prepared in Example 1, (2) (final concentration=6.25 μg/mL IgG^{Alexa488}, D'MEM, FBS free) were added to 2×10⁵ of HeLa cells in the D'MEM medium retained on a 6-well plate, and incubation was performed at 37° C. for 10, 15, 30, 45, 60, or 120 minutes. The cells were washed with a 20 U/mL cold heparin solution, then with the D'MEM medium containing FBS, and again with the 20 U/mL cold heparin solution. The washed cells were subjected to flow cytometry analysis using FACSan and CellQuest software (both are from Becton Dickinson). The analysis was performed in duplicate for 10,000 cells as the total cell count of each type of cells. The results are shown in FIG. 1.

It was observed that when the GALA peptide coexisted on the outer surface of the outermost lipid membrane, the efficiency for incorporation of the liposomes into cells was slightly reduced in a GALA peptide modification amount dependent manner. Further, from the viewpoint of the efficiency for incorporation into cells, it was observed that use of CHEMS was advantageous compared with use of PA.

(2) Confirmation of Intracellular Localization with Confocal Laser Scanning Microscopy

According to the method described in Example 1, multilamellar liposomes having a composition of DOPE:CHEMS:Chol-GALA=9:2:0.22 (molar ratio) in which IgG^{Alexa488} was bonded to the outer surface of the outermost lipid membrane (DCG2IgG) were prepared. DCG2IgG is a control liposome having the GALA peptide, but not having the R8 peptide.

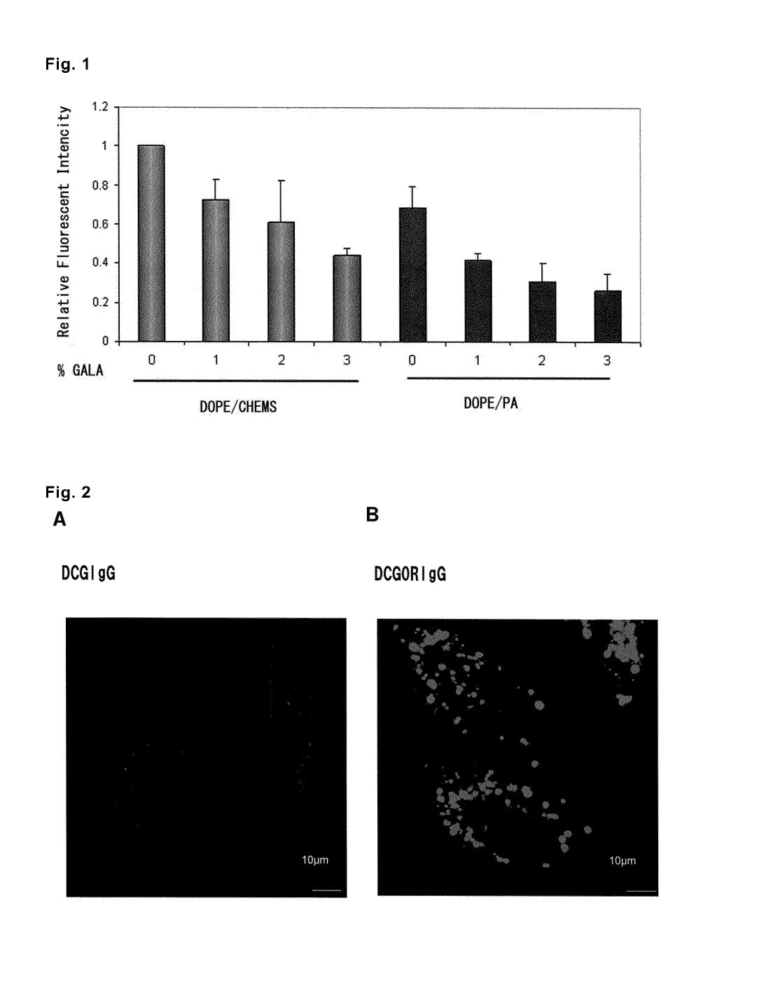

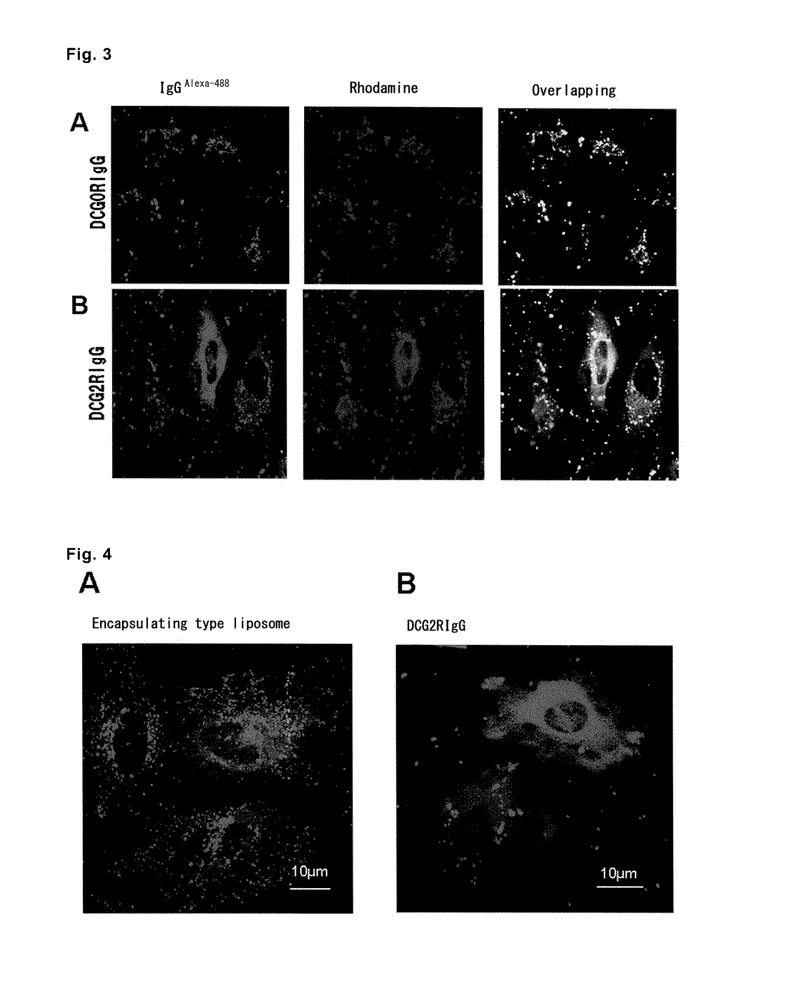

To 5×10⁴ of the HeLa cells in the D'MEM medium contained in a 35 mm glass bottom dish, DCG2IgG, as well as DCG0RIgG and DCG2RIgG prepared in Example 1, (2) (for all the liposomes, final concentration was 3.125 μg/mL IgG^{Alexa488}, D'MEM, FBS free) were separately added, and incubation was performed at 37° C. for 1 hour. The cells were washed with a 40 U/mL cold heparin solution, then the D'MEM medium containing FBS was added to the cells, and incubation was performed at 37° C. for 60 minutes. Immediately after the cell were washed again, intracellular localization of the liposomes (lipids modified with rhodamine) and IgG^{Alexa488} was investigated by using a laser scanning microscopy (Nikon A1 Confocal Imaging System, NIKON). The results are shown in FIGS. 2 and 3.

As shown in FIG. 2A, it was observed that DCG2IgG not having the R8 peptide was hardly incorporated into the HeLa cells, and as shown in FIG. 2B, IgG^{Alexa488} of DCG0RIgG not having the GALA peptide was trapped by endosomes in the inside of HeLa cells, and was not released into the cytoplasm. Similarly, whereas IgG^{Alexa488} of DCG0RIgG was trapped by the endosomes in the inside of the HeLa cells, and was not released into the cytoplasm (FIG. 3A), it was observed with DCG2RIgG that the fluorescence originating in IgG^{Alexa488} had spread over the whole cytoplasm except for the nucleus (FIG. 3B). From these results, it was confirmed that the liposomes of the present invention were incorporated into cells by endocytosis, and the lipids of the liposomes remained in the endosomes, while the antibodies bonding to the surfaces were released and spread in the inside of the cytoplasm.

### Example 3

### Comparison with Encapsulating Type Liposomes for Antibody-Releasing Ability

Liposomes encapsulating IgG^{Alexa488} (encapsulating type liposomes) were prepared in the same manner as that of Example 1, except that 250 μL of 10 mM HEPES buffer (pH 7.4) containing IgG^{Alexa488} was used in the hydration of Example 1, (1) instead of 250 μL of 10 mM HEPES buffer (pH 7.4).

According to the method described in Example 2(2), the encapsulating type liposomes were allowed to be incorporated into the HeLa cells, and intracellular localization of IgG^{Alexa488} was investigated. The results are shown in FIG. 4.

As shown in FIG. 4, when the encapsulating type liposomes were used, it was observed that most of fluorescence originating in IgG^{Alexa488} introduced into cells distributed in endosomes in a dot pattern (FIG. 4A). This result indicates that the antibodies enclosed in the encapsulating type liposomes were not released from the liposomes, but they remained to be trapped in the endosomes together with the liposomes. On the other hand, when the liposomes of the present invention were used, it was observed that the fluorescence originating in IgG^{Alexa488} more uniformly spread in the inside of the cytoplasm compared with the case of using the encapsulating type liposomes.

### Example 4

### Comparison with Commercial Available Protein-Introducing Reagents

A protein-introducing reagent sold by ACTIVE MOTIF, Chariot (registered trade name, http://www.activemotif.jp/catalog/37.html), in a volume of 100 μL (0.12 mg/mL) was mixed with 100 μL of 0.01 mg/mL of IgG^{Alexa488} in 10 mM HEPES buffer (pH 7.4) at room temperature for 30 minutes according to the conditions recommended by the manufacturer to prepare a Chariot-based introducing reagent.

Further, a protein-introducing reagent sold by Thermo Scientific, Pro-Ject (registered trade name, http://www.funakoshi.co.jp/node/10301), was reconstructed in chloroform according to the conditions recommended by the manufacturer, fractionated in a unit volume of 10 μL, and then dried. To the dried product, 0.05 mg/mL of IgG^{Alexa488} in 10 mM HEPES buffer (pH 7.4) was added, and the mixture was incubated for 10 minutes, and then subjected to ultrasonication to prepare a Pro-Ject-based reagent.

IgG^{Alexa488} was introduced into 5×10⁴ of the HeLa cells by using 40 μL of the Chariot-based introducing reagent or 40 μL of the Pro-Ject-based introducing reagent according to the conditions recommended by the manufacturer. Further, IgG^{Alexa488} was also introduced into 5×10⁴ of the HeLa cells by using DCG2RIgG (final concentration 3.125 μg/mL IgG^{Alexa488}, D'MEM, FBS free) of the present invention in the same manner as that of to the incorporation experiment described in Example 2(1). The incorporation abilities of two kinds of the aforementioned protein-introducing reagents, and the same of DCG2RIgG, which is the liposome of the present invention, were compared by flow cytometry analysis performed by using FACSan and CellQuest software (both are from Becton Dickinson). The results are shown in FIG. 5.

As shown in FIG. 5, it was observed that the introduction efficiency for IgG^{Alexa488} observed with the liposomes of the present invention markedly exceeded the introduction efficiencies observed by using each of Chariot (registered trade name) and Pro-Ject (registered trade name) under the recommended conditions.

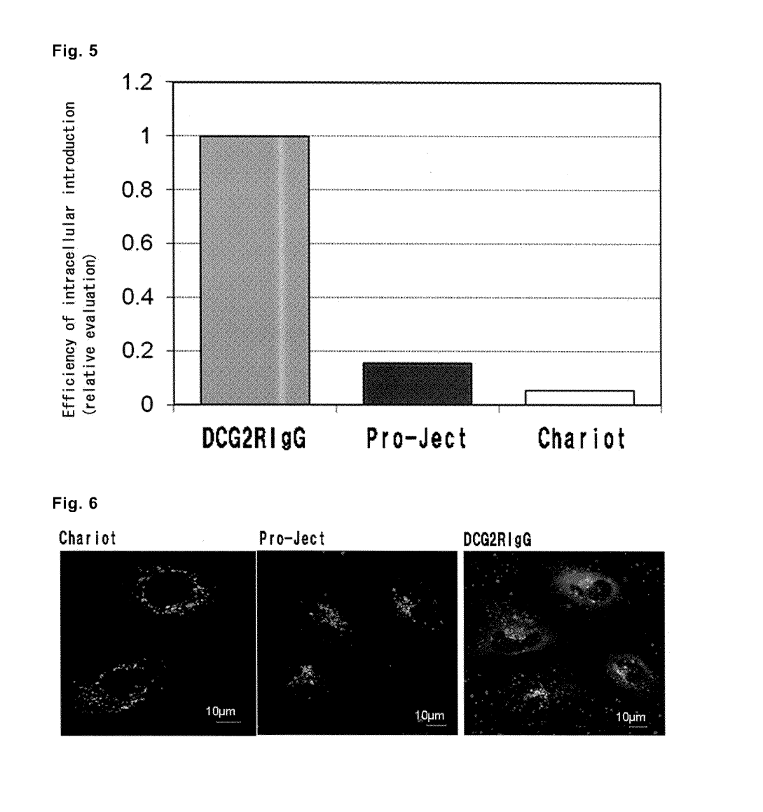

Further, localization of the introduced IgG^{Alexa488} in the HeLa cells was observed after the aforementioned various introductions of IgG^{Alexa488} with a confocal laser scanning microscopy according to the method described in Example 2(2). As a result, it was confirmed that both Chariot (registered trade name) and Pro-Ject (registered trade name) were trapped in the endosomes, and were not released into the cytoplasm (FIG. 6).

### Example 5

### Confirmation of Releasing Rate of Introduced Protein

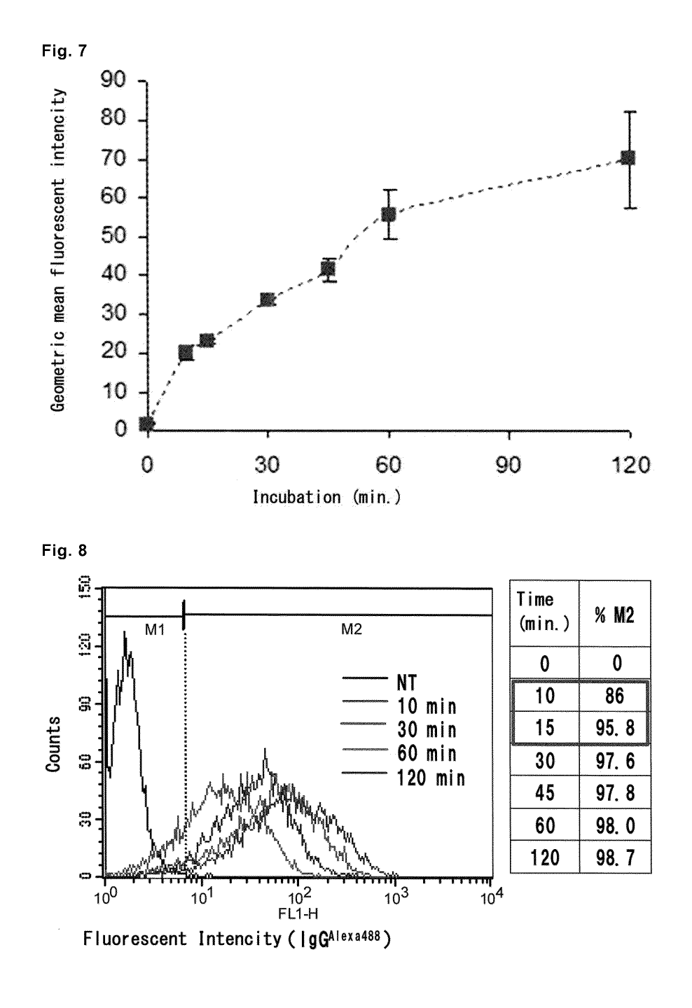

To 5×10⁴ of the HeLa cells in the D'MEM medium contained in a 35 mm glass bottom dish, DCG2RIgG prepared in Example 1(2) (final concentration 3.125 μg/mL IgG^{Alexa488}, D'MEM, FBS free) was added, the mixture was incubated at 37° C. for 10, 15, 30, 45, 60, or 120 minutes, then the cells were washed with a 40 U/mL cold heparin solution, and a part of the cells were subjected to flow cytometry analysis performed by using FACSan and CellQuest software (both are from Becton Dickinson). Further, for another part of the cells, intracellular localization of IgG^{Alexa488} was immediately investigated by using a confocal laser scanning microscopy (Nikon A1 Confocal Imaging System, NIKON). Furthermore, after the cells were washed with PBS, the cell nuclei were stained by adding Hoechst 33342 (final concentration, 1 μg/mL) and incubating the mixture for 5 minutes. The results are shown in FIGS. 7 to 9.

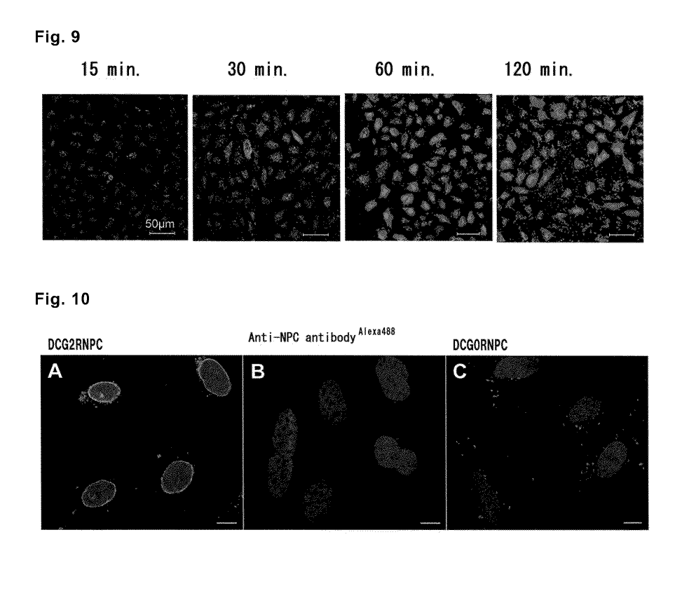

As shown in FIG. 7, it was observed that the intracellular incorporation amount of DCG2RIgG increased in proportion to the incubation time. Further, from the results shown in FIG. 8, it can be understood that the number of the cells in the M2 region (corresponding to cells that had sufficiently incorporated IgG^{Alexa488}) increased over time, and not less than 80% of the cells and not less than 95% of the cells were in the M2 region after incubations of 10 minutes and 15 minutes, respectively. This result indicates that the liposomes of the present invention are quickly incorporated into the cells. Furthermore, as shown in FIG. 9, release of IgG^{Alexa488} into the cytoplasm already started 10 minutes after the introduction of the liposomes, and release of the antibodies into the cytoplasm was observed in most of the cells 30 minutes after the introduction. It is considered that, with such reagents as Chariot (registered trade name) and Pro-Ject (registered trade name), it generally requires 3 to 4 hours to release antibodies into the cytoplasm. Therefore, it was confirmed that the liposomes of the present invention can provide superior releasing rate of a functional protein into the cytoplasm.

### Example 6

### Confirmation of Specific Binding Ability of Introduced Antibody

According to the method described in Example 1, liposomes DCG2RNPC were prepared, which liposomes correspond to the liposomes of the present invention, DCG2RIgG, in which IgG^{Alexa488} was replaced with the same amount of mouse anti-nuclear pore complex (NPC) antibody (IgG). The intracellular distribution of the anti-NCP antibody was detected by immunostaining using goat anti-mouse IgG^{Alexa488}.

To 5×10⁴ of the HeLa cells in the D'MEM medium contained in a 35 mm glass bottom dish precoated with gelatin, DCG2RNPC (final concentration 3.125 μg/mL anti-NPC antibody, D'MEM, FBS free) was added, and the mixture was incubated at 37° C. for 1 hour. After the cells were washed with a 40 U/mL cold heparin solution, the D'MEM medium containing FBS was added, and incubation was performed at 37° C. for 4 hours. The cells were fixed with cold ethanol at −20° C. for 7 minutes, then a 1% BSA solution was added, and incubation was performed at 37° C. for 30 minutes. Then, the solution was replaced with 10 ng/mL goat anti-mouse IgG^{Alexa488} in 1% BSA (secondary antibody), and incubation was performed at 37° C. for 1 hour. After the cells were washed with PBS, Hoechst 33342 (final concentration, 1 μg/mL) was added, and incubation was performed for 5 minutes to stain the cell nuclei. As controls, the HeLa cells to which the anti-NPC antibody solution was added as it was, and the HeLa cells for which the same operation as mentioned above was performed by using the liposomes DCG2RIgG of which anti-NPC antibodies were replaced with the same amount of nonspecific IgG were prepared. Intracellular localization of the anti-NPC antibodies in the aforementioned three kinds of the HeLa cells including the controls were observed by using a confocal laser scanning microscopy (Nikon A1 Confocal Imaging System, NIKON), and the results are shown in FIG. 10.

As shown in FIG. 10, it was observed that, in the HeLa cells to which DCG2RNPC was added, the fluorescence of the secondary antibodies binding to the anti-NPC antibodies existed along with the contour of the cell nucleus stained in blue by Hoechst 33342. Whilst, the fluorescence of the secondary antibodies binding to the anti-NPC antibodies was not observed at all in the HeLa cells to which the anti-NPC antibodies were directly added. In the HeLa cells to which DCG2RIgG was added, the fluorescence of the antibodies was confirmed in the cells in a dot pattern, and thus it was suggested that the nonspecific IgG remained to be trapped by the endosomes.

As described above, it was confirmed that the liposomes of the present invention could efficiently deliver antibodies into cells, and spread them in the cytoplasm with maintaining the specific binding ability of the antibodies.

### Example 7

### Stability of Carrier Liposome

The carrier liposome suspension of Example 1(1) was left at room temperature, and the particle size and ζ-potential thereof were measured over time by using the same method as that used in Example 1(2). As a result, it was observed that the carrier liposomes could maintain a shape having a size of 100 to 200 nm in a charged state over one month or longer. Further, the liposomes of the present invention, DCG2RIgG, were prepared from the stored carrier liposomes, and the incorporation ability thereof was confirmed according to the method of Example 2(2). As a result, the antibodies were introduced into the cells at efficiency comparable to that of DCG2RIgG observed in Example 2.

### Example 8

### Introduction of Anti-P-Akt Antibody and Anti-ATAT3 Antibody

DCG2RPAkt and DCG2RPSTAT3, which are the liposomes of the present invention, were prepared in which the mouse anti-nuclear pore complex (NPC) antibodies (IgG) used in Example 6 was replaced with either of mouse anti-P-Akt (phosphorylated Akt) antibodies (Millipore) or rabbit anti-STAT3 (Signal Transducer and Activator of Transcription 3) antibody (Millipore).

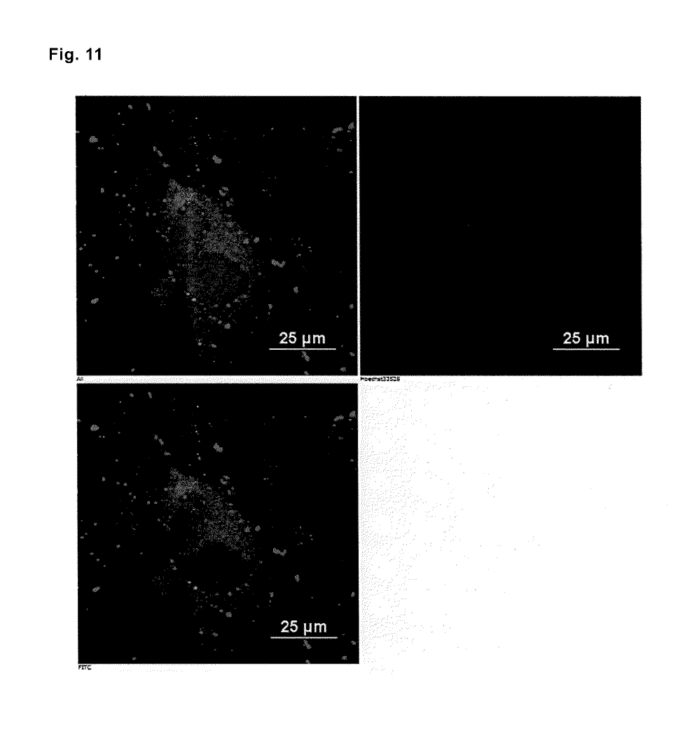

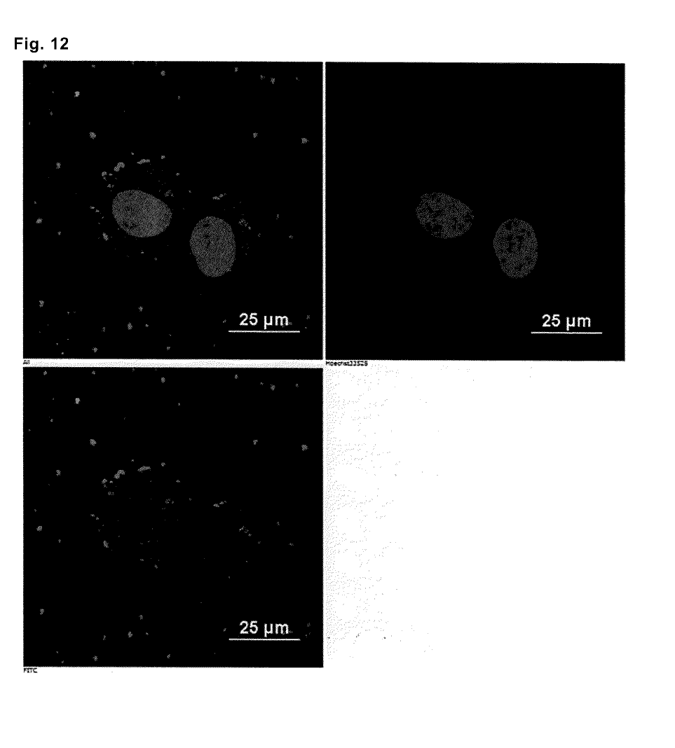

To the DMEM medium (containing antibiotics and FBS) retained on an 8-well chamber (Nunc), the HeLa cells were inoculated in an amount of 7.5×10³ cells/well, and cultured for 24 hours. The medium was removed, DCG2RPAkt or DCG2RPSTAT3 (final concentration 3.125 μg/mL of each type of antibodies, D'MEM, FBS free) washed with the DMEM medium (antibiotics free, containing FBS) was added, and the mixture was incubated at 37° C. for 1 hour. The medium was replaced with the D'MEM medium containing FBS, incubation was further performed at 37° C. for 1 hour, and then the cells were fixed with paraformaldehyde (PFA) (4% PFA, 15 minutes, room temperature). Then, after the cells were washed 3 times with PBS(−), 0.1% Triton was added, and incubation was performed at room temperature for 10 minutes. The cells were further washed 3 times with PBS(−), a 1% BSA solution was added, and incubation was performed at 37° C. for 30 minutes. Then, the cells were washed 3 times with PBS(−), 10 ng/mL of goat anti-rabbit IgG^{Alexa488} (Invitrogen, for detection of STAT3) or 10 ng/mL of goat anti-mouse IgG^{Alexa488} (Invitrogen, for detection of P-Akt) was added, and incubation was performed at 37° C. for 1 hour. The cells were observed by using a confocal laser scanning microscope (Nikon A1 Confocal Imaging System, NIKON) under the microscope setting conditions of Objective lens Plan Apo 60×/1.20 PFS WI, First Dichroic Mirror (405/488/561/640). Further, nuclear staining was performed by using Hoechst 33342 (blue). Intracellular localization of each type of the antibodies in the HeLa cells that incorporated them was observed, and the results are shown in FIG. 11 (DCG2RPAkt) and FIG. 12 (DCG2RPSTAT3).

For the anti-P-Akt antibodies, localization of the antibodies (green) was observed in the cytoplasm and cell membrane, in which P-Akt is considered to exist, and for the anti-STAT3 antibodies, localization of the antibodies (green) was observed in the cytoplasm, in which STAT3 is considered to exist.

### Example 9

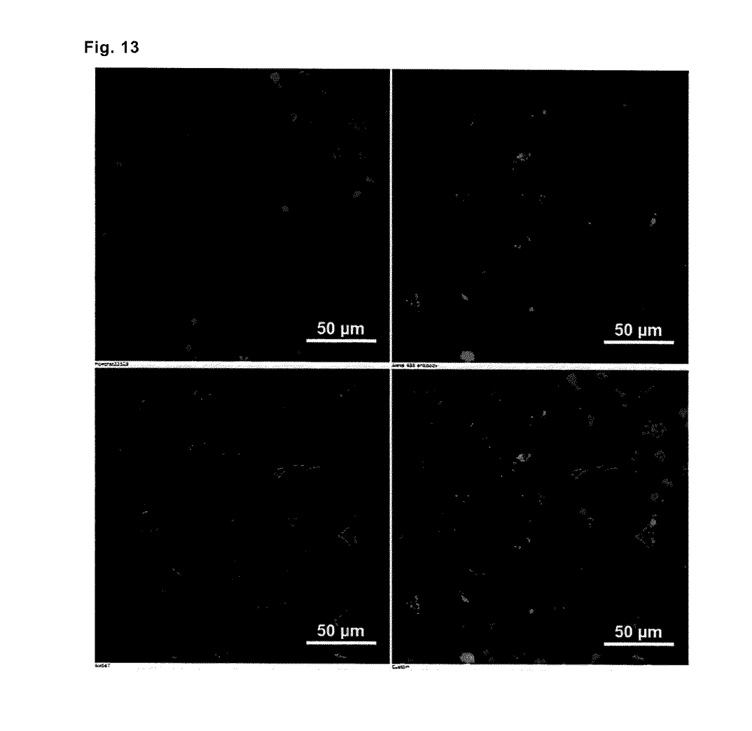

The liposomes DCG2RIgG of the present invention prepared in Example 1(2) were administered to a C57BL/6J about mouse (CLEA, Tokyo, Japan) from the caudal vein. The liver was collected 30 minutes after the administration, and accumulation of the DCG2RIgG in the liver was observed be using a confocal laser scanning microscopy (Nikon A1 Confocal Imaging System, NIKON). The observation was performed under the microscope setting conditions under Objective lens Plan Apo 60×/1.20 PFS WI, First Dichroic Mirror (405/488/561/640). Further, nucleus staining was performed with Hoechst 33342, and staining of liver vessel endothelial cells was performed with Alexa 647-conjugated isolectin (Invitrogen). The results are shown in FIG. 13. As shown in FIG. 13, it was observed that IgG (green of Alexa 488) administered together with DCG2RIgG was delivered to the liver tissue (especially liver vessel endothelial cells, red of Alexa 647) under the aforementioned conditions.

### INDUSTRIAL APPLICABILITY

The liposome of the present invention enables quick and highly efficient delivery of a functional protein non-covalently binding to an outer surface of a lipid membrane into a cell. Therefore, the liposome is useful, because it can intracellularly deliver an antibody directed to a biological molecule existing in a cell as a target, or a functional protein that interacts with such a biological molecule with maintaining the physiological functions thereof.

1. A liposome comprising a lipid covalently bonded with a polyarginine peptide consisting of 4 to 20 continuous arginine residues and a lipid covalently bonded with a GALA peptide consisting of the amino acid sequence of SEQ ID NO: 1 and/or an R-GALA peptide consisting of the amino acid sequence of SEQ ID NO: 2 as component lipids of lipid membrane, and having a lipid membrane in which a protein to be intracellularly delivered is non-covalently bound to outer surface of the membrane.

2. The liposome according to claim 1, wherein the polyarginine peptide, the GALA peptide and/or the R-GALA peptide, and the protein to be intracellularly delivered are disposed on outer surface of one lipid membrane.

3. The liposome according to claim 1, wherein the protein to be intracellularly delivered is a specific antibody directed to an intracellular protein.

4. The liposome according to claim 1, which is a single layer liposome comprising one lipid membrane.

5. The liposome according to claim 1, which comprises two or three lipid membranes.

6. The liposome according to claim 1, which is for delivering a protein having a molecular weight larger than 1 kDa into cytoplasm or an intracellular organelle.

7. The liposome according to claim 2, wherein the protein to be intracellularly delivered is a specific antibody directed to an intracellular protein.

8. The liposome according to claim 2, which is a single layer liposome comprising one lipid membrane.

9. The liposome according to claim 3, which is a single layer liposome comprising one lipid membrane.

10. The liposome according to claim 2, which comprises two or three lipid membranes.

11. The liposome according to claim 3, which comprises two or three lipid membranes.

12. The liposome according to claim 2, which is for delivering a protein having a molecular weight larger than 1 kDa into cytoplasm or an intracellular organelle.

13. The liposome according to claim 3, which is for delivering a protein having a molecular weight larger than 1 kDa into cytoplasm or an intracellular organelle.

14. The liposome according to claim 4, which is for delivering a protein having a molecular weight larger than 1 kDa into cytoplasm or an intracellular organelle.

15. The liposome according to claim 5, which is for delivering a protein having a molecular weight larger than 1 kDa into cytoplasm or an intracellular organelle.
